(19) United States
(12) Patent Application Publication
(10) Pub. No.: US 2010/0099734 A1
(43) Pub. Date: Apr. 22, 2010

(54) AMINO ACID AND ITS USES

(75) Inventors: Andre Vosloo, Potchefstroom (ZA); Leon Van Rensburg, Potchefstroom (ZA)

Correspondence Address: SCHWEGMAN, LUNDBERG & WOESSNER, P.A., P.O. BOX 2938, MINNEAPOLIS, MN, 55402, US

(73) Assignee: North-West University, Potchefstroom (ZA)

(21) Appl. No.: 11/992,178

(22) PCT Filed: Sep. 19, 2006
(86) PCT No.: PCT/IB2006/053353

§ 371 Date: Dec. 8, 2009

(30) Foreign Application Priority Data
Sep. 20, 2005 (ZA) 2005/07602

(51) Int. Cl. A61K 31/401; C07D 207/04; A61P 43/00

(52) U.S. Cl. 514/423; 548/532

(57) ABSTRACT

This invention relates to a supplement, the use thereof and method for supplementing the concentration of free cellular L-proline in an organism, for restricting dehydration of such an organism, the supplement comprising an effective amount of free L-proline. The supplement is effective in reducing the levels of free radicals in organisms experiencing water stress. This invention further relates to a method of treating dehydration in humans and animals including the step of administering to an individual in need thereof an effective amount of a supplement according to the third aspect of the invention. An effective amount of free L-proline is typically between 20 and 1000 mg, preferably 100 mg free L-proline per kilogram body mass of the organism, three times a day.

### INTRODUCTION AND BACKGROUND

This invention relates to an amino acid and its uses. This invention further relates to compositions and methods for treating humans and animals and the use of such an amino acid.

Water is an essential molecule for sustaining life and an animal's ability to cope with changes in its internal water content is paramount for its survival. Loss of internal water is a threat common to all animals. Such losses could occur through evaporation, waste excretion, or osmosis. Osmosis usually occurs in a saline environment, such as sea water, or by extracellular freezing, or from diseases such as diabetes or hyperglycemia, causing osmotic imbalances.

In humans, loss of water occurs through breathing, sweating, urinating and other processes. This loss of water is exacerbated during exercise, high temperature and dry environmental conditions. As a result of water loss and oxidative stress, levels of intracellular free radicals increase, causing damage to the cells, and if this process is not curbed, it is most often fatal.

Taurine is a sulfur-based, non-protein amino acid and is known to act as an osmolyte in cases of dehydration. Taurine is also said to be cytoprotective by acting as an antioxidant, a calcium modulator, a synaptic neuromodulator and a membrane stabilizer. For this reason, it is known to include taurine in energy drinks for human consumption.

It is known to use L-proline orally in addition to other compositions such as ceramides and fish cartilage hydrolysate in the maintenance of the correct skin hydration as disclosed in EP 1 514 554 A1. However, L-proline has thus far not been recognized as an antioxidant or free radical scavenger.

It has been shown that L-proline concentration increases when freshwater and brackish water animals are exposed to hyperosmotic stress. It is also known that L-proline is released from cells of Australian Abalone (Haliotis roei) to maintain cell volume at low salinities. L-proline is therefore generally considered as an intracellular osmolyte of animal cells, but to date, most studies were focused on the intracellular production and secretion of L-proline in cases where the organism is placed under hyperosmotic stress. However, a disadvantage of the prior art is that none of the published prior art studies is directed at the use of free L-proline supplementation in the prevention and/or treatment of dehydration and increase in free radicals in organisms placed under hyperosmotic stress.

EP 1 514 554 A1 discloses a composition based on natural substances, useful in the maintenance of the correct skin hydration and in the prevention and/or the treatment of the effects of skin ageing, in particular wrinkles, characterized in that it comprises in combination ceramides in the form of extract of Oryza sativa; fish cartilage hydrolysate; and one or more amino acids selected from L-proline, L-leusine, L-valine and L-cysteine. This composition comprises between 20 to 200 mg, preferably 40 mg per day of these amino acids. A disadvantage of this composition is that the levels of L-proline that are suggested are insufficient to elevate the concentration of cellular free L-proline to the extent that it restricts dehydration and reduction in free radical concentration in the user, when exposed to water and heat stress.

### OBJECTS OF THE PRESENT INVENTION

It is accordingly an object of the present invention to provide a supplement and method with which the aforesaid disadvantages could be alleviated and to provide new uses of L-proline.

### SUMMARY OF THE INVENTION

According to a first aspect of the invention there is provided use of an effective amount of free L-proline in the preparation of a supplement for supplementing the concentration of free cellular L-proline in an organism, for restricting dehydration of such an organism.

The supplement may be prepared by providing between 20 mg and 1000 mg free L-proline per kilogram body mass of the organism to benefit from the use thereof, in an oral administration form.

The supplement may be prepared by mixing 100 mg free L-proline per kilogram body mass of the organism to benefit from the use thereof, with a drink suitable for human consumption.

According to a second aspect of the invention there is provided use of an effective amount of free L-proline as a supplement for supplementing the concentration of free cellular L-proline in an organism, for restricting dehydration of such an organism.

The supplement may comprise between 20 mg and 1000 mg free L-proline per kilogram body mass of the organism to benefit from the use thereof, in an oral administration form.

The supplement may comprise 100 mg free L-proline per kilogram body mass of the organism to benefit from the use thereof, mixed with a drink suitable for human consumption.

According to a third aspect of the invention there is provided a supplement for supplementing the concentration of free cellular L-proline in an organism, for restricting dehydration of such an organism, the supplement comprising an effective amount of free L-proline.

The above supplement may be effective in reducing the levels of free radicals in organisms experiencing water stress.

The effective amount of L-proline may comprise between 20 mg and 1000 mg free L-proline per kilogram body mass of the organism to benefit from the use thereof, in an oral administration.

The effective amount of L-proline may comprise 100 mg free L-proline per kilogram body mass of the organism to benefit from the use thereof, mixed with a drink suitable for human consumption.

According to a fourth aspect of the invention there is provided a method of treating dehydration in humans and animals including the step of administering to an individual in need thereof an effective amount of a supplement according to the third aspect of the invention.

In one embodiment the organism is a human and the free L-proline concentration is supplemented through oral administration of effective amounts of free L-proline to counter dehydration of such human. It will be appreciated that such supplementation would be particularly required and effective in cases where the human to whom the supplement is administered experience water stress owing to exercise, heat or lack of water intake. It will be appreciated further that the supplement would be effective in increasing free L-proline concentration and water levels in such a human, where the supplement is taken prophylactically.

According to a fifth aspect of the invention there is provided a method of supplementing the concentration of free cellular L-proline in an organism, for restricting dehydration of such an organism including the step of administering to such organism an effective amount of free L-proline.

An effective amount of free L-proline may be between 20 mg and 1000 mg, preferably 100 mg free L-proline per kilogram body mass of the organism, three times a day.

### EXAMPLE

An effective amount of a supplement according to a preferred embodiment of the invention for restricting dehydration and for reducing the cellular concentration of free radicals in humans, was prepared by mixing 100 mg free L-proline per kilogram body mass of the human with any conventional off-the-shelf energy or cold drink.

Therefore, for an adult male with a mass of 80 kg, the supplement was prepared by mixing 8 g of free L-proline with 500 ml energy drink. The supplement was taken orally by a adult male three times a day.

It was found that use of the supplement lead to an increase in cellular concentration of free L-proline in the user and the elevated concentration was so maintained.

It was surprisingly further found that the supplement substantially reduced water loss and dehydration of the user, particularly when exposed to heat and water stress and during exercising.

It is foreseen that the supplement according to the invention could find wide application in the field of preventing and treating dehydration in humans and animals such as the use in the prevention of dehydration in extreme athletes; the use as supplement to hydration drips in human patients (including pediatric application); the use to enhance feeds for ornamental fish prior to international shipment; and the use enhancing feeds of sheep, cattle, poultry, fish and other animals prior to slaughtering and freezing.

It will be appreciated that variations in detail are possible with the invention without departing from the scope of the appended claims.

1. Use of an effective amount of free L-proline in the preparation of a supplement for supplementing the concentration of free cellular L-proline in an organism, for restricting dehydration and reducing the levels of free radicals of such an organism.

2. Use according to claim 1 wherein the supplement is prepared by providing between 20 mg and 1000 mg free L-proiine per kilogram body mass of the organism to benefit from the use thereof, in an oral administration form.

3. Use according to claim 2 wherein the supplement is prepared by mixing 100 mg free L-proline per kilogram body mass of the organism to benefit from the use thereof, with a drink suitable for human consumption.

4. Use of an effective amount of free L-proiine as a supplement for supplementing the concentration of free cellular L-proline in an organism, for restricting dehydration of such an organism.

5. Use according to claim 4 wherein the supplement comprises between 20 mg and 1000 mg free L-proline per kilogram body mass of the organism to benefit from the use thereof, in an oral administration form.

6. Use according to claim 5 wherein the supplement comprises 100 mg free L-proline per kilogram body mass of the organism to benefit from the use thereof, mixed with a drink suitable for human consumption.

7. A supplement for supplementing the concentration of free cellular L-proline in an organism, for restricting dehydration of such an organism, the supplement comprising an effective amount of free L-proline.

8. (canceled)

9. A supplement according to claim 7 wherein the effective amount of L-proline comprises between 20 mg and 1000 mg free L-proline per kilogram body mass of the organism to benefit from the use thereof, in an oral administration form.

10. A supplement according to claim 7 wherein the effective amount of L-proline comprises 100 mg free L-proline per kilogram body mass of the organism to benefit from the use thereof, mixed with a drink suitable for human consumption.

11. A method of treating dehydration in humans and animals including the step of administering to an individual in need thereof an effective amount of a supplement according to claim 7.

12. A method according to claim 11 wherein the organism is a human and the free L-proline concentration is supplemented through oral administration of effective amounts of free L-proline to counter dehydration of such human.

13. A method according to claim 12 wherein the human to whom the supplement is administered experiences water stress owing to exercise, heat or lack of water intake.

14. A method according to claim 11 wherein the supplement is taken prophylactically and is effective in increasing free L-proline concentration and water levels in the human.

15. A method of supplementing the concentration of free cellular L-proline in an organism, for restricting dehydration of such an organism including the step of administering to such organism an effective amount of free L-proline.

16. A method according to claim 9 wherein the effective amount of free L-proline is between 20 mg and 1000 mg, per kilogram body mass of the organism to benefit therefrom, three times a day.

17. A method according to claim 10 wherein the effective amount of free L-proline is 100 mg free L-proline per kilogram body mass of the organism, three times a day.

18. Use of free L-proline in the preparation of a supplement substantially as herein described and exemplified.

19. Use of free L-proline as a supplement substantially as herein described and exemplified.

20. A supplement substantially as herein described and exemplified.

21. A method of treating dehydration substantially as herein described and exemplified.

22. A method of supplementing the concentration of free cellular L-proline in an organism substantially as herein described and exemplified.
